(19) United States
(12) Patent Application Publication
(10) Pub. No.: US 2005/0015002 A1
(43) Pub. Date: Jan. 20, 2005

(54) Integrated protocol for diagnosis, treatment, and prevention of bone mass degradation

(76) Inventors: Gary S. Dixon, Milwaukee, WI (US); Andreas M. Pfander, Torrance, CA (US)

Correspondence Address: JONES DAY, 555 WEST FIFTH STREET, SUITE 4600, LOS ANGELES, CA, 90013-1025, US

(21) Appl. No.: 10/623,466

(22) Filed: Jul. 18, 2003

(51) Int. Cl. A61B 5/00; A61B 8/00

(52) U.S. Cl. 600/407; 600/449; 600/595

(57) ABSTRACT

The present invention provides improved methods and systems for diagnosis, prevention, and treatment of a bone related condition such as osteoporosis. The present invention integrates bone mass measurement techniques with various preventive and treatment measures to create a protocol for prevention and treatment of the bone related condition. In one embodiment, a medical practitioner treats bone mass degradation occurring in a patient by measuring a bone characteristic level in at least one of the patient's bones to yield a score, conducting a gait analysis to yield a gait characterization, measuring a bone mass marker concentration in at least one of the patient's body fluids to yield a bone marker level, and prescribing one or more therapies. Optionally, the treatment may include designating a future time to repeat the measurement of a bone characteristic level, the gait analysis, and the measurement of bone marker level.

### FIELD OF THE INVENTION

The present invention relates generally to the treatment of bone diseases and, more specifically, to methods and systems for diagnosis, treatment, and prevention of ailments related to the loss of bone mass.

### BACKGROUND

Bone mass deterioration is a widespread medical condition, appearing with particular frequency in the elderly and in women. The gradual depletion of a person's bone mass can make the bone prone to fracture and/or deformation and cause numerous accompanying adverse effects, including pain and discomfort. One condition, known as osteoporosis, manifests itself as a decrease in bone tissue mass and often leads to fractures of the vertebrae, hip, femur, and distal end of the wrist bone.

The World Health Organization defines osteoporosis as comprising four diagnostic categories, normal, osteopenia, osteoporosis, and established osteoporosis, and further defines those categories using diagnostic value ranges. Currently, within the United States, osteoporosis affects about 20-25 million people. Osteopenia, a condition where a patient has a lower than normal bone density, afflicts 16% of white women aged 20-29. Within that demographic, less than 1% have osteoporosis. Approximately 38%of women aged 65 have osteopenia while 20%have osteoporosis and, by age 80, the percentage of women with normal bone density decreases to 15%. The percentages depend on race, age, and hormone usage. Due to this condition, one out of every six women will have a hip fracture and one out of every three women will have a vertebral fracture during their lifetime.

A person may be at risk of having osteoporosis, or at risk of some degree of bone loss or low bone mass, based upon his or her age, sex, medical history, lifestyle, or family medical history. Specifically, an exemplary set of risk factors that may be used to identify people whose bone mass should be assessed include vertebral compression fracture, age greater than 65 years, family history of osteoporotic fracture, fragility fracture after age 40, malabsorption syndrome, systemic glucocorticoid therapy of more than 3 months, primary hyperparathyroidism, tendency to fall, osteopenia apparent on x-ray film, hypogonadism, and menopause before the age of 45. Other risk factors include past history of clinical hyperthyroidism, rheumatoid arthritis, excessive caffeine intake, low dietary calcium intake, smoking, chronic anticonvulsant therapy, excessive alcohol intake, weight less than 125 lbs., weight loss that is greater than 10% of total weight at the age of 25, and chronic heparin therapy.

Certain medical evaluations can be conducted to determine whether osteoporosis may be present in a patient, including the examination of a patient's height and weight, investigating the presence of pain or deformity in the bones, and identifying underlying medical illnesses using blood cell counts, PTH blood tests, mineral content (calcium, phosphorus, among others), a thyroid test, and vitamin D levels. Once major deterioration has occurred, it is difficult to restore the lost bone. Thus, therapeutic efforts must be directed towards early recognition of the progressive disease so that treatment can be instituted before irreversible structural damage occurs.

One approach to diagnosing the existence of osteoporosis in a patient or a patient's susceptibility to bone-loss related ailments, such as bone fractures or osteopenia, is to test a patient's bone and compare the values to established references. Various devices may be used. Ultrasound techniques are advantageous in that they are non-invasive and operate on the principle that the velocity and attenuation of the signal through the patient's bone is a measure of the characteristics of the bone. For treatment purposes, relying solely on the measurement of bone characteristics to compare against established references is disadvantageous because patients often have to wait for a long time to ascertain whether bone formation or resorption is occurring.

Another method of diagnosing the deterioration of bone mass is by using biochemical markers indicative of bone turnover. Whenever bone formation or resorption occurs, various chemical reactions occur within the body, which elevate the presence of certain indicators in the body fluids, referred to as biochemical markers, indicating changes in the bone status and, consequently, indicating a greater or lower rate of bone formation or resorption. Using biochemical markers, however, also has considerable disadvantages. It provides little practical information for estimating BMD level. Furthermore, biochemical markers are present in tissues other than bone and can be influenced by non-skeletal processes. Also, unlike densitometers, biochemical markers do not provide information about a specific bone or body regions. Thus, biochemical markers cannot independently be used to diagnose bone depletion and predict facture risk.

Certain systems provide for a biochemical bone measuring unit and a densitometric bone measuring unit to form a bone measuring system that performs biochemical and densitometric assessments of bone material. The system provides practitioners with bone characteristic data to evaluate bone status, and in some instances provides a prognosis as to future bone characteristics. In one embodiment, the system combines the biochemical bone measuring unit and the densitometric bone measuring unit into a single housing. In an alternative embodiment, the densitometric and biochemical units are connected to each other via data communication circuitry and either the densitometric bone measuring unit or the biochemical bone measuring unit has a controller that combines the measurements from each unit to provide bone characteristic data. In another embodiment, the biochemical bone measuring unit and the densitometric bone measuring unit may be individual units that separately perform biochemical and densitometric bone assessments.

Despite coupling a bone density measuring and bone marker measuring system, the abovementioned systems have significant disadvantages. Specifically, they merely provide for the use of known measurement systems without providing any type of protocol or method for how to practically integrate the various measurements in a holistic diagnosis and treatment paradigm.

Certain protocols do exist for the diagnosis and treatment of osteoporosis. For example, it is recommended that 1) persons over the age of 65 should have a BMD test; 2) persons over the age of 50 with at least one major, or two minor, risk factors should have a BMD test; 3) postmenopausal women with risk factors for fracture should have a BMD test; 4) higher intakes of calcium and vitamin D are recommended, particularly in adults over 50 (calcium 1500 mg/day and vitamin D 800 IU/day); and 5) people should participate in exercise, particularly weight-bearing exercises such as brisk walking, running or dancing. Formal protocols, such as the Osteoporosis Risk Assessment Instrument (ORAI) and Simple Calculated Osteoporosis Risk Estimation (SCORE), provide more defined algorithms for identifying persons at risk for osteoporosis based on variables such as the person's age, weight, and estrogen use.

However, to properly initiate, conduct, and monitor the effects of a treatment and/or prevention regimen, sufficient knowledge of the state of a person's bone mass, along with rate of increase or decrease is preferred. Current treatment and/or prevention protocols fail to adequately account for or incorporate such information.

Although exercising, dietary, and other methods of prevention may exist, there is a need to integrate these various preventive and/or treatment measures with bone measurement techniques to create an integrated osteoporosis treatment protocol. There is also a need for improved methods and systems to determine changes in bone mass in a short period of time, to examine patients and analyze bone deformities to comprehensively assess bone material, and to provide a practitioner with bone data to predict future bone characteristics, to prevent bone loss, to avoid fractures, and to increase bone density.

### SUMMARY OF THE INVENTION

The present invention provides improved methods and systems for the diagnosis, prevention, and treatment of osteoporosis. The present invention integrates bone mass measurement techniques with various preventive and treatment measures to create a protocol for the prevention and treatment of a bone related condition such as osteoporosis. Further, the present invention allows for the specific targeting of persons at risk for fracture or bone mass degradation while not requiring mass screening of individuals, thereby providing an efficient and cost-effective approach to osteoporosis for the medical community.

In one embodiment, a medical practitioner treats a bone related condition occurring in a patient by measuring a bone characteristic in the patient's bone to yield a first score, such as a T-score; conducting a gait analysis to yield a gait characterization; measuring a bone marker concentration in at least one of the patient's body fluids to yield a bone marker level; and prescribing a therapy based on at least one of the measurement of a bone characteristic level, the gait analysis and the measurement of a bone mass marker concentration. Optionally, the treatment may include designating a future time to repeat the measurement of the bone characteristic, the gait analysis, and the measurement of bone marker level. Further, the steps of measuring a bone characteristic level, conducting a gait analysis and measuring a bone marker concentration may be performed in any order.

The bone characteristic may be measured using a bone characteristic measuring unit that comprises a space for housing a portion of the patient, a positioning device for holding the portion, a plurality of ultrasound transducers for transmitting and detecting signals, and an output for outputting the bone characteristic measurement score value. Optionally, the bone characteristic is measured using X-ray absorptiometry (dual or single), quantitative ultrasonometry, or quantitative computed tomography.

Preferably, the score utilized in the present invention is a T-score, as determined from a value measured by the bone characteristic measurement unit. The therapy may be prescribed based upon an output of an integrated unit having received the T-score value, the gait characterization, and the bone marker level value. Further optionally, the integrated unit comprises a receiver in data communication with a processing unit and a display unit in data communication with the processing unit. Optionally, the present invention further comprises the step of determining a likelihood of a patient injuring at least one of the patient's bones. Optionally, the bone marker level is measured by a bone marker measurement device that comprises a container containing a body fluid, a mechanism for holding the said container, an analyzer for determining a concentration of an absorbing constituent in a solution, and an output for outputting the bone marker level value.

Optionally, the gait is characterized by a gait analysis procedure conducted on a patient wherein the procedure comprises the steps of examining the balance of the patient wherein the patient is standing on both feet, examining the balance of the patient wherein the patient is standing on a first foot, and examining the balance of the patient wherein the patient is standing on a second foot.

Optionally, a patient's risk factors are assessed to help determine the therapy. The therapy may be one of recommending life style changes, recommending weight bearing exercises, recommending resistance exercises, recommending increasing calcium intake, recommending increasing vitamin D intake, and recommending at least one of bisphosphonates, calcitonin, estrogen replacement therapy, and raloxifene.

Optionally, with respect to the future times for measurement repeats, the present invention includes, within a first pre-defined time period, re-measuring a bone characteristic in at least one of the plurality of bones to yield a second score having a value; within a second pre-defined time period, re-conducting a gait analysis to yield a second gait characterization; and within a third pre-defined time period, re-measuring a bone marker concentration in at least one body fluid of the patient to yield a second bone marker level having a value. The present invention may further include the step of comparing the first T-score to the second T-score, the first gait characterization to the second gait characterization, and the first bone marker level to the second bone marker level, and prescribing a therapy based upon at least one of the comparisons. Further, the first, second and third periods may differ.

In another embodiment, the present invention is a system for treating bone related condition of a patient, comprising a bone characteristic measurement unit having an output for communicating a bone characteristic level value, a gait analysis unit having an output for communicating a gait characterization, and a bone marker measurement unit having an output for communicating a bone marker level value.

Optionally, the bone characteristic measurement unit comprises a space for housing a portion of said patient, a positioning device connected to said chamber for holding said portion, a plurality of ultrasound transducers for transmitting and detecting signals, and an output for outputting the bone characteristic level value. Optionally, the bone marker measurement unit comprises a container containing a body fluid, an analyzer for determining a concentration of an absorbing constituent in a solution, and an output for outputting the bone marker level value.

In another embodiment, the present invention is a method for treating a bone related condition of a patient comprising the steps of instructing a medical practitioner to measure a bone characteristic level in at least one of the plurality of bones to yield a score having a value, based upon the value of the score, instructing the medical practitioner to conduct a gait analysis to yield a gait characterization, based upon the value of the score and the gait characterization, instructing the medical practitioner to measure a bone marker concentration in at least one body fluid of the patient to yield a bone marker level having a value, providing the medical practitioner with a plurality of therapies that can be prescribed, and instructing the medical practitioner to designate a future time to repeat the measurement of a bone characteristic level, the gait analysis, and the measurement of bone marker concentration.

In another embodiment, the present invention is a method for treating a bone related condition of a patient comprising the steps of measuring a bone characteristic of a bone of a patient to yield a T-score having a value; if the T-score is abnormal, conducting a gait analysis to yield a gait characterization; if the gait characterization is abnormal, measuring a bone marker concentration in at least one body fluid of the patient to yield a bone marker level having a value; prescribing a therapy; and designating a future time to repeat the measurement of a bone characteristic level, the gait analysis, and the measurement of bone marker concentration.

The future time to repeat the measurement of a bone characteristic level may be during the twelfth month from the previous measurement. The future time to repeat the gait analysis may include scheduling a series of eight gait analyses over a period of time. The future time to repeat the bone marker measurement may be during the third month from the previous measurement.

These and other embodiments shall be described in reference to the drawings and the detailed description.

### BRIEF DESCRIPTION OF DRAWINGS

These and other features and advantages of the present invention will be further appreciated, as they become better understood by reference to the following detailed description when considered in connection with the accompanying drawings:

FIG. 1a is a flowchart depicting data flow for one embodiment of the present invention;

FIG. 1b is a flowchart depicting a process of one embodiment of the present invention;

FIG. 1c is a flowchart depicting a process of another embodiment of the present invention;

FIG. 1d is a flowchart depicting a process of another embodiment of the present invention;

FIG. 1e is a flowchart depicting a process of another embodiment of the present invention;

FIG. 1f is a flowchart depicting a process of another embodiment of the present invention;

FIG. 2 is perspective view of one embodiment of a bone characteristic measuring unit;

FIG. 3 is a block diagram illustrating one embodiment of circuitry used in connection with one embodiment of a bone density measuring unit;

FIG. 4 depicts one method of assaying bone markers using a plate well;

FIG. 5 depicts an exemplary reaction of a label enzyme with a substrate during a labeled immunoassay technique;

FIG. 6 provides a perspective view of one embodiment of a bone marker measuring unit;

FIG. 7 provides a schematic view of one embodiment of a gait analysis unit; and

FIG. 8 is a graph of T-scores relative to percentage of population.

### DETAILED DESCRIPTION

The present invention provides a protocol for assessing bone characteristics and recommending a treatment regimen using bone characteristic, bone marker, and gait analysis data and existing therapies such as vitamin and mineral supplements, exercise routines, lifestyle modifications, and drug therapies. The present invention will be described with reference to aforementioned drawings. One of ordinary skill in the art would appreciate that the applications described herein are examples of how the broader concept can be applied, that the methods and systems provided herein may be used by a medical practitioner, care-giver, or other health care provider, and that the methods and systems provided herein may be further taught to medical practitioners.

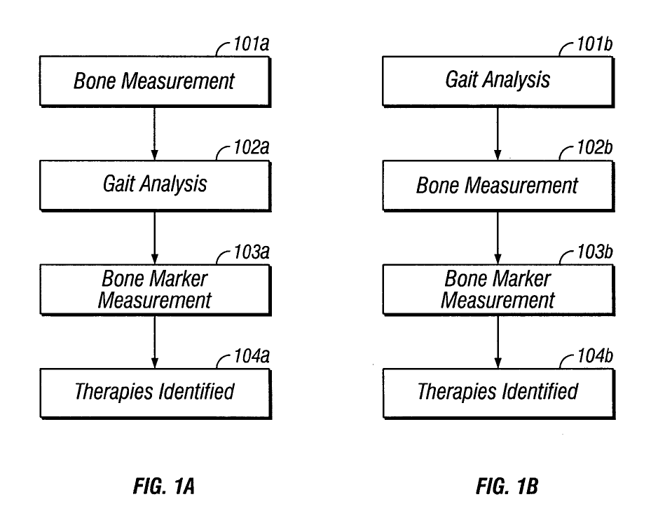

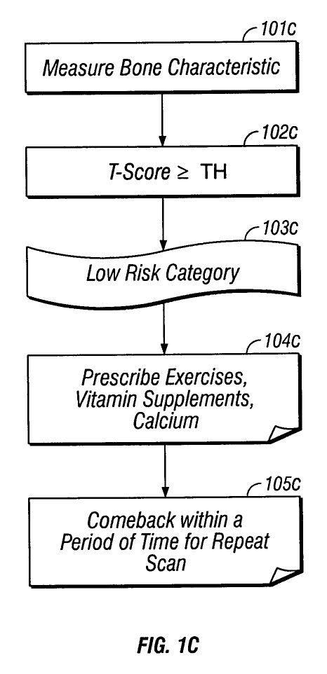

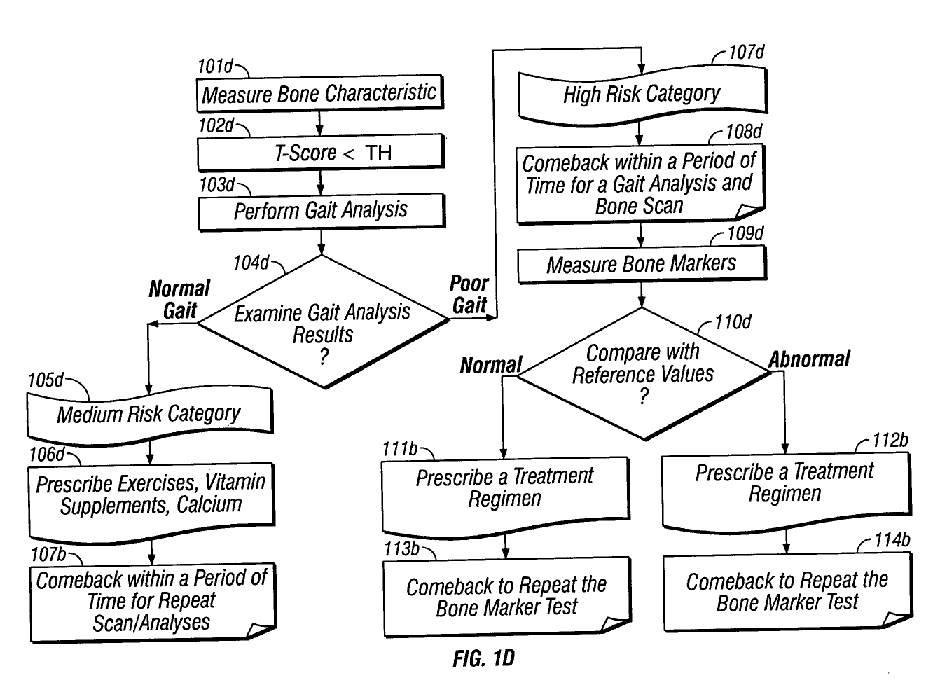

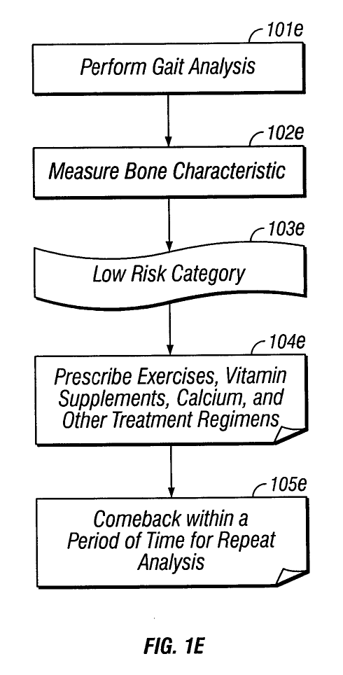

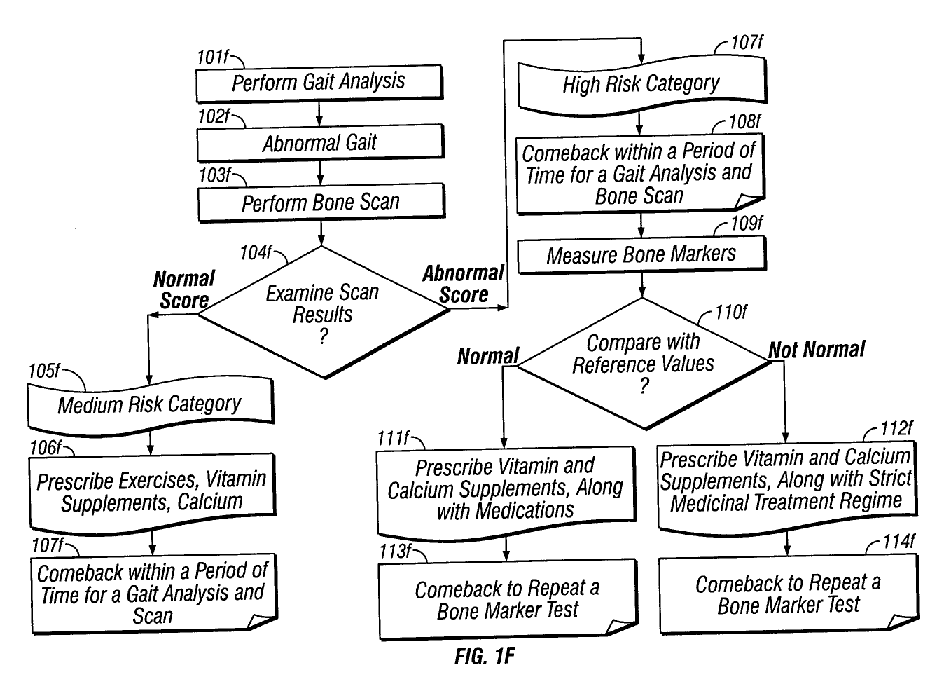

Referring to FIG. 1a, data flow for one embodiment of the present invention is shown. A patient is first examined with the bone characteristic measuring unit 101a to obtain values from which certain scores, such as the T-score, will be derived. The gait of the patient is then analyzed using a gait analysis unit and/or gait analysis procedure 102a, to assess body imbalance. The level of bone turnover or resorption markers is then determined using the bone marker measuring unit 103a. Finally, prevention and treatment therapies are prescribed 104a. In another embodiment, as shown in FIG. 1b, the gait of the patient is analyzed using a gait analysis unit and/or gait analysis procedure 101b, to assess body imbalance. A patient is then examined with the bone characteristic measuring unit 102b to obtain values from which certain scores, such as the T-score, will be derived. The level of bone turnover or resorption markers is then determined using the bone marker measuring unit 103b. Finally, prevention and treatment therapies are prescribed 104b.

As further described below, one of ordinary skill in the art would appreciate that the order and use of each unit may be dependent upon the data, results, and findings generated in other units, that subsequent diagnoses and tests are scheduled and performed depending on the results obtained herein, and that treatment therapy may vary according to the extent of bone loss as determined by the various methods of diagnosis. For example, if the score measured by the bone density measuring unit is above the required level, bone marker testing and gait analysis may not be performed and a standard prevention therapy may be prescribed. Similarly, if the gait is found to be normal but the score measured by the bone density measuring unit yield abnormal results, the bone marker testing may still be performed and a particular therapy may be prescribed. FIG. 1c is a procedural flow diagram, associated with one embodiment of the invention, depicting a course of action when a patient's bone mineral density score, when compared to the appropriate reference value, is comparable to, or above, a corresponding threshold value. The score referred to herein refers to any known scoring method, protocol, or system for evaluating the bone mineral density of a patient. Although the term score is used interchangeably with the term T-score, it is recognized that a T-score is simply one type of score that may used in the present invention. Other scoring approaches, particularly those that are used or endorsed by health organizations, may be used to evaluate a patient's bone mineral density.

Referring back to FIG. 1c, the patient is examined 101c to determine the patient's T-score. If the T-score is, for example, equal to or above a pre-defined threshold “TH”, such as −1.0, 0 or a positive number, 102c, or is generally representative of a patient in a low risk category, the patient is classified 103c into the low fracture risk category. As part of the low fracture risk category, the patient may not be required to undergo any further tests. Accordingly, the appropriate exercises, calcium, vitamin D supplements, and other therapies and treatments may be prescribed 104c. The patient may further be advised 105c to come back within a period of time, preferably between 24-36 months or more preferably during the twenty-fourth month, for a second bone characteristic measurement. This process is repeatable throughout the life of a patient, thereby acting as a recurring check on the patient's bone mineral density that is performed periodically.

FIG. 1d is a procedural flow diagram, associated with another embodiment of the invention, illustrating the course of action in a second instance when the patient's T-score is below a corresponding threshold value, “TH”, such as −1.0, zero, or a positive number. The patient is examined 101d to determine the patient's T-score. If the T-score is, for example, below the threshold value 102d, indicating the patient has below normal bone mass, a gait analysis is performed 103d to ascertain the patient's balance 104d.

If the gait is normal, the patient is classified 105d into the medium fracture risk category. Optionally, a biochemical bone marker measurement may be taken to determine and record the patient's rate of bone formation. Accordingly, the medical practitioner recommends 106d one or more exercises, calcium, vitamin D supplements, and medications. The patient may be advised to comeback within a first period of time for a gait analyis and within a second period of time for a bone characteristic scan. For example, the patient may be advised to obtain a gait analysis between 9-15 months, or preferably during the twelfth month. The patient may also be advised to obtain a bone characteristic scan between 9-15 months or preferably during the twelfth month. Further, if applicable, the patient may be advised to obtain a bone marker test between 2-4 months or preferably during the third month. Preferably, the patient continues the treatment and testing regimen until an improvement in the T-score is achieved.

If the gait is poor and, therefore, indicative of an imbalance which could lead to a fall and possibly bone fractures, the patient is classified 107d into the high fracture risk category. Biochemical bone markers are then measured 109d and compared to and expected range or reference values 110d. Where the bone marker concentrations indicate a normal condition, the patient may be prescribed 111d calcium, vitamin D supplements, exercise, other regimens, and medications. The patient may further be advised to comeback within a first period of time for a gait analysis 108d, a second period of time for a bone marker analysis 113b, and a third period of time for a bone characteristic scan 108d. For example, the patient may be advised to obtain a gait analysis between 1-4 months and preferably during the second month. Alternatively, the patient may be placed on a gait analysis schedule that involves performing a gait analysis once every two weeks for sixteen consecutive weeks, or preferably, performing a gait analysis once a week for eight consecutive weeks. Where the patient is placed on a gait analysis schedule, the patient may have, for example, a medical practitioner conduct the gait analysis. Alternatively, the patient may perform a self-gait analysis using, for example, a pressure sensing platform device, which will be described in further detail below, and report the result to the medical practitioner.

The patient may also be advised to obtain a bone characteristic scan between 9-15 months or preferably during the twelfth month. Further, the patient may be advised to obtain a bone marker test between 2-4 months or preferably during the third month. Preferably, the patient continues the treatment and testing regimen until an improvement in the marker level, gait, and/or T-score is achieved.

Alternatively, where the bone marker concentrations indicate a borderline or abnormal condition, the patient may be prescribed 112b certain calcium and vitamin D supplements along with strict medicinal treatment regime. The patient may further be advised to comeback within a first period of time for a gait analysis 108d, a second period of time for a bone marker analysis 114b, and a third period of time for a bone characteristic scan 108d. For example, the patient may be advised to obtain a gait analysis between 1-4 months and preferably during the second month. Alternatively, the patient may be placed on a gait analysis schedule that involves performing a gait analysis once every two weeks for sixteen consecutive weeks, or preferably, performing a gait analysis once a week for eight consecutive weeks. Where the patient is placed on a gait analysis schedule, the patient may have, for example, a medical practitioner conduct the gait analysis. Alternatively, the patient may perform a self-gait analysis using, for example, a pressure sensing platform device, which will be described in further detail below, and report the result to the medical practitioner.

The patient may also be advised to obtain a bone characteristic scan between 9-15 months or preferably during the twelfth month. Further, the patient may be advised to obtain a bone marker test between 2-4 months or preferably during the third month. Preferably, the patient continues the treatment and testing regimen until an improvement in the marker level, gait, and/or T-score is achieved.

Referring to FIG. 1e, a procedural flow diagram, associated with another embodiment of the invention, is shown. A gait analysis 101e is performed to ascertain the patient's balance and propensity to fall and be susceptible to bone fractures. Next, the patient's T-score is examined 102e. If the gait analysis and the T-score is determined to be normal, the patient is classified 103e into a low risk fracture category. Accordingly, the medical practitioner may recommend 104e one or more exercises, calcium, vitamin D supplements, medications, and other treatments or therapies. The patient may further be advised 105e to comeback within a period of time for a gait analysis and a bone characteristic scan. For example, the patient may be advised to obtain a gait analysis and a bone characteristic scan after 24 months. This process is repeatable throughout the life of a patient, thereby acting as a periodic check on the patient's condition.

If the T-score examined at 102e is below a threshold value, the patient is classified into a medium fracture risk category. A biochemical bone marker measurement is also taken to record the patient's rate of bone formation. Based on part or all of the measured values or analysis results, the medical practitioner may recommend one or more exercises, calcium, vitamin D supplements, and medications. The patient may be advised to come back within a first period of time for a gait analysis, a second period of time for a bone characteristic scan, and a third period of time for a bone marker analysis. For example, the patient may be advised to obtain a gait analysis between 9-15 months, or preferably during the twelfth month. The patient may also be advised to obtain a bone characteristic scan between 9-15 months, or preferably during the twelfth month. Further, the patient may be advised to obtain a bone marker test between 2-4 months or preferably during the third month. This process is repeatable and is preferably continued until an improvement in the T-score is achieved.

Referring to FIG. 1f, a procedural flow diagram, associated with another embodiment of the invention, is shown. A gait analysis 101f is performed to ascertain the patient's balance and propensity to fall and be susceptible to bone fractures. If the gait is determined to be abnormal 102f, the patient is examined 103f to determine the patient's T-score. If the T-score is equal to or above the threshold value 104f, indicating the patient at least has normal bone mass, the patient is categorized in a medium risk category 105f and a medical practitioner may recommend 106f one or more exercises, calcium, vitamin D supplements, medications, therapies, and treatments. The patient may further be advised 107f to comeback within a first period of time for a gait analysis and within a second period of time for a bone characteristic scan. For example, the patient may be advised to obtain a gait analysis between 1-4 months and preferably during the second month. Alternatively, the patient may be placed on a gait analysis schedule that involves performing a gait analysis once every two weeks for sixteen consecutive weeks, or preferably, performing a gait analysis once a week for eight consecutive weeks. Where the patient is placed on a gait analysis schedule, the patient may have, for example, a medical practitioner conduct the gait analysis. Alternatively, the patient may perform a self-gait analysis using, for example, a pressure sensing platform device, which will be described in further detail below, and report the result to the medical practitioner.

The patient may also be advised to obtain a bone characteristic scan between 24-36 months or preferably during the twenty-fourth month. Preferably, the patient continues the treatment and testing regimen until an improvement in the gait is achieved.

If the T-score is below the threshold value 104f, indicating the patient has below normal bone mass, the patient is categorized 107f into a high risk category. Biochemical bone markers are then measured 109f and compared to reference values 110f. Where the bone marker concentrations indicate a normal condition, the patient may be prescribed 111f calcium, vitamin D supplements, exercise, other regimens, and medications. The patient may further be advised to comeback within a first period of time for a gait analysis 108f, a second period of time for a bone marker analysis 113f, and a third period of time for a bone characteristic scan 108f. For example, the patient may be advised to obtain a gait analysis between 1-4 months and preferably during the second month. Alternatively, the patient may be placed on a gait analysis schedule that involves performing a gait analysis once every two weeks for sixteen consecutive weeks, or preferably, performing a gait analysis once a week for eight consecutive weeks. Where the patient is placed on a gait analysis schedule, the patient may have, for example, a medical practitioner conduct the gait analysis. Alternatively, the patient may perform a self-gait analysis using, for example, a pressure sensing platform device, which will be described in further detail below, and report the result to the medical practitioner. The patient may also be advised to obtain a bone characteristic scan between 9-15 months or preferably during the twelfth month. Further, the patient may be advised to obtain a bone marker test between 2-4 months or preferably during the third month. Preferably, the patient continues the treatment and testing regimen until an improvement in the marker level, gait, and/or T-score is achieved.

Alternatively, where the bone marker concentrations indicate a borderline or abnormal condition, the patient may be prescribed 112f certain calcium and vitamin D supplements along with a medicinal treatment regime. The patient may further be advised to comeback within a first period of time for a gait analysis 108f, a second period of time for a bone marker analysis 114f, and a third period of time for a bone characteristic scan 108f. For example, the patient may be advised to obtain a gait analysis between 1-4 months and preferably during the second month. Alternatively, the patient may be placed on a gait analysis schedule that involves performing a gait analysis once every two weeks for sixteen consecutive weeks, or preferably, performing a gait analysis once a week for eight consecutive weeks. Where the patient is placed on a gait analysis schedule, the patient may have, for example, a medical practitioner conduct the gait analysis. Alternatively, the patient may perform a self-gait analysis using, for example, a pressure sensing platform device, which will be described in further detail below, and report the result to the medical practitioner. The patient may also be advised to obtain a bone characteristic scan between 9-15 months or preferably during the twelfth month. Further, the patient may be advised to obtain a bone marker test between 2-4 months or preferably during the third month. Preferably, the patient continues the treatment and testing regimen until an improvement in the marker level, gait, and/or T-score is achieved.

The present invention further contemplates and covers processes that performs a bone mineral density analysis, gait analysis and/or a bone marker analysis irrespective of whether the first analysis performed yields a normal result. Moreover, the present invention covers processes whereby the bone measuring process, the gait analysis and the bone marker measuring process may be sequenced in any suitable order. For example, the bone marker test may be performed first followed by a bone mineral density test and the gait analysis.

Furthermore, the present invention contemplates and covers processes whereby the second or subsequent bone characteristic measurement(s) may or may not be taken from the same bone that was examined previously. However, it is preferred to measure the bone characteristic from the same bone and to use the same machine or type of machine to minimize variation in the collected data.

As provided in greater detail below, the present invention utilizes a plurality of measurement techniques to provide methods and systems designed to help medical practitioners, such as doctors, nurses, technicians, chiropractors, and other health care professionals, diagnose and treat osteoporosis. Because osteoporosis is an endemic condition, the whole body of a patient is generally affected by bone degradation. Accordingly, it is possible to predict the risk of injuring one bone, for example the hip bone, by examining or measuring the bone characteristic of another bone, for example the heel bone. Combining these diagnostic tests increases the likelihood of identifying bone mass degradation in one of a plurality of bones of a patient early in the process, preventing bone fractures or other injuries, and stabilizing or reversing the bone loss process. The present invention further helps cost-effectively address bone loss related ailments by selecting high risk individuals and avoiding mass screening or unnecessary examination.

A plurality of bone mass measurement devices exist that can be used to determine a patient's bone characteristic. X-ray based systems operate on the principle that bone attenuates or absorbs ionizing radiation and, therefore, the bone characteristic, which is referred to as bone mineral density, can be determined based upon the amount of radiation that passes from a X-ray source, through the bone, and into a radiation detector. In one embodiment of the present invention, the bone mass measurement unit comprises a device employing singe energy X-ray absorptiometry (SXA). SXA uses an X-ray tube to produce a singe photon beam directed at a body part immersed in a water bath to simulate a uniform soft-tissue thickness. SXA is effectively used to image distal skeletal sites, such as the calcaneus, and typically generates bone mineral density measurements in terms of grams per centimeter squared (g/cm²).

In another embodiment of the present invention, the bone mass measurement unit comprises a device employing dual energy X-ray absorptiometry (DXA). DXA measurements can be performed at central sites, such as the spine and hip, or at peripheral sites, such as the forearm, calcaneus, or wrist and typically generates bone mineral density measurements in terms of grams per centimeter squared (g/cm²).

In another embodiment of the present invention, the bone mass measurement unit comprises a device employing quantitative computed tomography (QCT). QCT generates an image of a thin transverse slice through the body and measures true volumetric bone density (e.g., a three-dimensional measurement expressed in g/cm³) derived from tissue attenuation measurements. Because attenuation is dependent on tissue density and composition, QCT allows for distinct measurements of both trabecular and cortical bone density of several sites in the body. QCT is available in either a single-energy mode or dual-energy mode, which has a higher radiation dose. One of ordinary skill in the art would appreciate that other photon radiation based bone measurement approaches exist, including radiographic absorptiometry and singe and dual photon absorptiometry.

X-ray based systems have, however, several disadvantages. They are often relatively expensive, require a large amount of operational space, and lack portability. Moreover, because X-ray devices emit ionizing radiation, they may require a licensed technician to operate the equipment, limiting the range of users.

In a preferred embodiment, quantitative ultrasonometry (QUS) is used to measure a patient's bone characteristic, which is referred to as either a quantitative ultrasound index (QUI) or stiffness index (SI), by, for example, measuring the propagation of an ultrasound pulse through the patient's heel. As opposed to X-ray based systems, QUS does not rely on ionizing radiation. Instead, it uses broadband ultrasound attenuation (BUA), which is a measure of the attenuation of the ultrasound pulse through the bone, and speed of sound (SOS), which is a measure of the time the sound pulse takes to travel through the heel. Because the velocity of sound is higher in healthy bone, QUS can measure bone mass and give some information about bone microarchitecture. More specifically, in patients with osteoporosis, the attenuation of the sound wave is reduced and the SOS value is smaller, thereby affecting both the BUA and SOS values. QUS is typically conducted on the patient's heel, finger and/or tibia.

In one embodiment, because the speed of sound is dependent upon the degree of connectivity of the trabeculae, the SOS value can be used to evaluate the connectivity and elasticity of bone. The speed of the ultrasonic acoustic signal is measured at a number of frequencies at multiple locations. Typically, normal bone has higher SOS than osteoporotic bone because of better linkage.

Additionally, because the attenuation of ultrasound is dependent upon bone structure, the BUA value can be used to evaluate bone density and obtain some information about bone structure. The attenuation of the ultrasonic acoustic signal is measured at one or more frequencies at multiple locations. Typically, normal bone has higher attenuation than osteoporotic bone because of its rigid composition. The BUA may then be calculated as the slope of the attenuation as a function of the ultrasonic frequency.

To evaluate the strength, structure, and mineral content of a patient's bones, and therefore, whether the individual is suffering from insufficient bone density, some ultrasound densitometers combine BUA and SOS measurements to determine the quantitative bone characteristic from which a T-score is determined. Certain QUS systems generate a quantitative ultrasound index (QUI) or stiffness index (SI), which are ratios of the BUA value to the SOS value and are considered equivalents to bone mineral density measurements. One of ordinary skill in the art would appreciate that other combinations of BUA and SOS can be used to determine bone mineral density measurements. According to the World Health Organization (WHO), a T-score is defined as the number of standard deviations from the average bone density value of young (25-30 year old) individuals of the same sex and ethnicity. One of ordinary skill in the art would appreciate that the value of the T-score provides a relative assessment of how much greater, or lower, the patient's bone density is as compared to the average bone density of a young individual. The T-score may be determined from a bone characteristic measurement, such as bone mineral density, quantitative ultrasound index, or stiffness index.

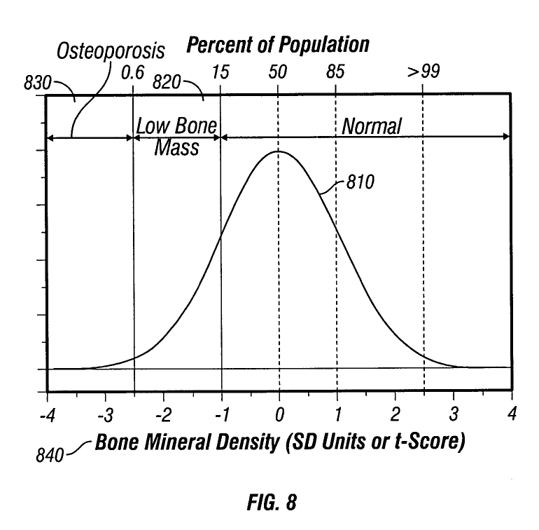

Medical practitioners can use the T-score to diagnose the existence of bone thinning or osteoporosis. Referring to FIG. 8, a T-score of above −1.0 810 indicates substantially no bone deterioration and the patient is normal. The patient may be defined as having a low bone density 820, referred to as osteopenia, if the T score is between −1.0 and −2.5. Finally, the patient may be defined as having a very low bone density and substantial bone loss 830, referred to as osteoporosis, if the T score is less than −2.5. Although the graph is presented in terms of standard deviations relative to a bone mineral density level, one of ordinary skill in the art would appreciate that similar graphs are applicable to other bone characteristic data, such as quantitative ultrasound index or stiffness index.

There are numerous ways to interpret bone characteristics measurements and medical practitioners may use different metrics for determining what is, and is not, significant bone loss warranting treatment. For example, if the bone characteristic is measured for multiple areas of a patient's body, thereby deriving multiple T-scores, certain health care providers may use the lowest T score to diagnose the patient. Therefore, if a T score of −3 were obtained at the hip and −2 were obtained at the arm, the doctor may use the −3 T score as a basis to conclude the patient is suffering from osteoporosis.

Additionally, there may be other ways to define a reference level against which to compare a patient's bone characteristic values and, therefore, other ways to represent the relative state of a patient's bone condition. For example, the bone characteristic data may also be used to determine a Z score, which is defined as the number of standard deviations from the average bone density value of individuals of the same age, sex, and ethnicity. The present invention is not limited to the specific reference definitions described herein.

While a plurality of different bone characteristic measurement devices may be used in the present invention, it should be noted that the bone characteristic data, and therefore the T-scores, generated in different devices may vary a great deal. Specifically, a patient examined with QCT may yield a lower T-score than QUS. Therefore, T-scores must be interpreted in light of the devices used.

T-scores must further be interpreted in light of which part of the body had been measured. The most commonly measured sites, the axial and appendicular skeleton, consist of the bone and cartilage in the head, neck, and trunk (axial) and the shoulder blade, collarbone, the upper and lower limbs, and the pelvis (appendicular). Peripheral areas of the appendicular skeleton are also measured and include the forearm, phalanges, os calcis, and most preferably calcaneus. Bone characteristic measurements of the axial or appendicular skeleton or of the peripheral areas can be useful in making a clinical decision regarding intervention for the prevention or treatment of osteoporosis.

Further, it should be noted that the bone characteristic measurement is preferably conducted in the context of a full physical exam so that the root causes for bone loss can be determined. In certain cases, low bone characteristic values may be caused by a plurality of other conditions, including hyperthyroidism, multiple myeloma, Cushing's syndrome, hyperparathyroidism, rickets, premature menopause, vitamin D deficiency, and ankylosing spondylitis.

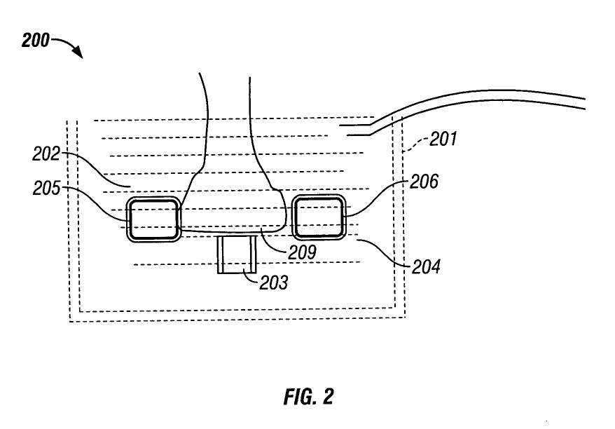

Referring to FIG. 2, a perspective view of the bone characteristic measuring unit of the present invention is shown. The bone characteristic measuring unit 200 comprises a region 201, reference liquid medium 202, positioning device 203, and ultrasound transducers 204 and 205. The region 201 contains a reference liquid medium 202 in which the patient's heel bone, or calcaneus, 209 is immersed. The positioning device 203 is provided to support the patient's calcaneus. The first ultrasound transducer 204 and the second ultrasound transducer 205 are positioned on either side of the patient's calcaneus 209 and are held by suitable supports not shown. The transducers 204 and 205 are connected by mechanical linkages to motors enabling them to scan a rectangular area generally corresponding to the portion of the calcaneus to be scanned. One of ordinary skill in the art would appreciate that there can be arrays of transducers for sending and receiving the ultrasound signals on both sides of the body portion being scanned. One of ordinary skill in the art would also appreciate that the bone characteristic measuring unit can comprise ultrasound transducers that are fixed in place and scan a singular area of the target scan region, such as the calcaneus.

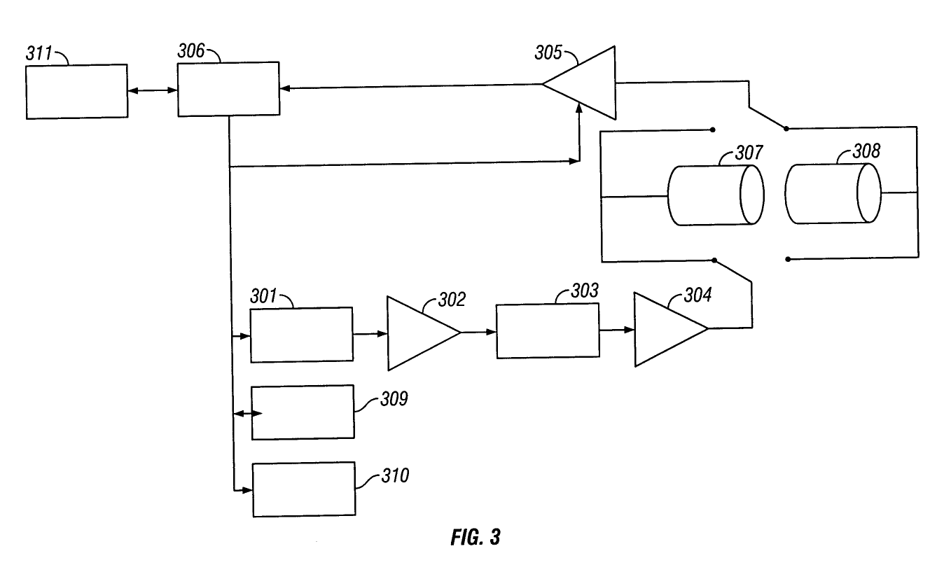

Referring to FIG. 3, the block diagram illustrating the circuitry used in connection with the above described bone characteristic measuring unit is shown. The circuitry 300 comprises digital analog converter 301, voltage controlled sine-oscillator (VCO) 302, signal control unit 303, power amplifier 304, receiver amplifier 305, digital signal processor (DSP) 306, transducers 307 and 308, motor control block 309, temperature probe 310, and display panel 311. The digital analog converter 301 supplies power to the VCO 302, which can produce signals having variable frequencies. The signal control unit 303 regulates these signals and feeds them to the transducers 307 and 308 via the power amplifier 304. The receiver amplifier 305 amplifies the signal received from the transducers, which is sampled and read into the DSP 306, which examines the signal and adjusts the gain. The motor control block 309 is used for positioning the transducers in the vertical and horizontal directions so that a selected area can be scanned by moving the transducers in the scanning pattern. The temperature probe 310 is used to register the temperature of the water or other reference liquid around the calcaneus.

Operationally, a scan is performed by moving the transducers 307 and 308 synchronously in the horizontal and vertical directions over an area of the area being scanned, most preferably the patient's calcaneus. While in motion, signals are emitted from the first transducer 307 and are received by the second transducer 308 in transmission mode and received back by the transmitting transducer in pulse echo or reflection mode. Attenuation is measured at each location at a desired number of frequencies, preferably in the range of 100 kHz to 1 MHz, more preferably between 200 and 600 kHz. Broadband ultrasonic attenuation (BUA) may then be calculated by the DSP 306 at each scanned location as the slope of the attenuation as a function of the ultrasound frequency. Speed of sound (SOS) is also calculated by the DSP 306. The DSP 306 then utilizes BUA and SOS to determine a value, such as QUI, SI, or BMD, from which the T-score can be derived.

The calcaneus is analyzed because that it has high content of spongy trabecular bone. Also, because of the prevalence of osteoarthritic changes in the central skeleton, measurements at the calcaneous provide a more accurate assessment because it is a weight bearing bone. Moreover, assessments of fracture risk at the calcaneus site are equally predictive of the fracture risk in the entire skeleton. However, any part of the body may be used, including the forearm or other appendages.

One of ordinary skill in the art would appreciate that the present invention can employ any type of densitometer, including varying designs for QUS, QCT, DXA, or SXA systems. One would further appreciate that the areas of the body that could be used to generate a T-score include any part of the patient's skeleton.

In a preferred embodiment, the T-score generated by measurements made with the densitometer is used together with the gait analysis data to identify an individual at high risk for bone fracture and to increase the specificity of estimated bone loss. Patients having a decreased bone mass have an increase fracture risk for both vertebral and nonvertebral sites, such as the wrist or hip. Because fracture risk is inversely proportional to bone density, for each standard deviation below the young adult peak mean bone mass, the risk of fracture increases up to three fold. The most common sites of osteoporotic fractures are the wrist, spine and hip. While most fractures can be resolved with surgery, hip fractures may prevent a person from walking independently and spine fractures may result in curvature of the spine (dowager's hump) or loss of height.

Gait analysis is conducted to inspect a patient's gait, namely the patient's particular manner of moving on foot, and generate a gait characterization. The measurements provide details on the bone joint angles/positions and relative risk for falling. A patient determined to have low or rapidly decreasing bone mass by the densitometer is analyzed using such a gait analysis system to further determine the patient's susceptibility to bone fractures. Patients with more negative T scores and imbalance during walking are at greater risk of breaking a bone during an accident or fall.

In one embodiment, the gait analysis is conducted by employing an observational approach. The individual is made to stand on both feet and the posture is analyzed for balance, stability, symmetry, and foot support pattern. Subsequently, the individual is made to stand on one foot at a time and again each stance is observed for the distribution of forces below the foot. Observational gait analysis is generally more reliable when it focuses on proximal segments instead of distal segments.

In a second embodiment, the gait analysis is conducted by employing a device having at least two platforms capable of sensing pressure. As known to those of ordinary skill in the art, a patient stands on the platforms, with one foot on a first platform and a second foot on a second platform, thereby exerting pressure on the two platforms. A lack of stability, symmetry, or foot support pattern can be determined by analyzing the pressure differential detected by the two platforms. The platforms can be pressures pads, scales, or other measurement devices. Further, these type of platform devices may be used at the patient home to allow the patient to perform a self-gait analysis.

To facilitate the patient to perform a self-gait test, these platform devices may be portable and be used in the patient's home.

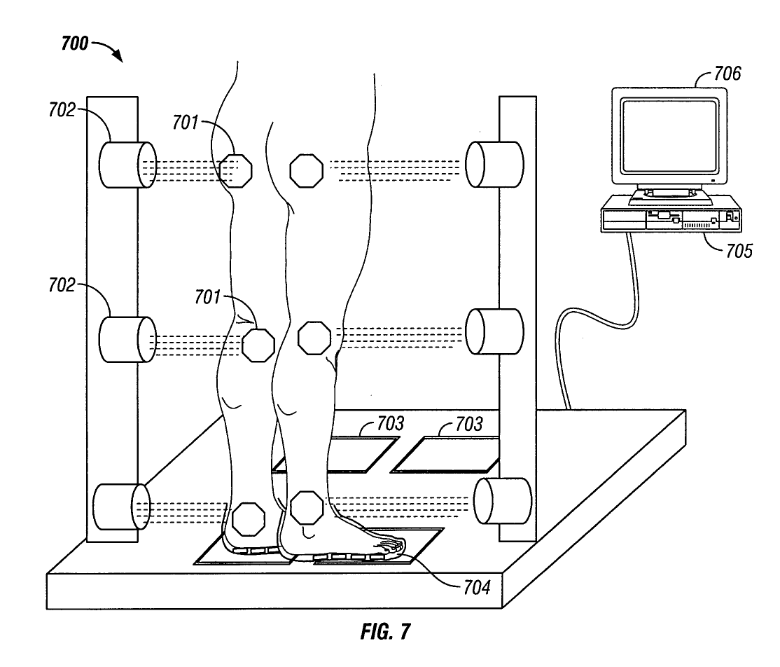

In another embodiment, shown in FIG. 7, the gait analysis system 700 includes detectors, such as electrogoniometers, 701, infrared motion cameras 702, force platforms 703, sensors 704, processing unit 705, and display panel 706. The electrogoniometers 701 are secured to the hip, knee, and ankle joints of both the legs of the patient and function as reflective markers during walking. The infrared motion cameras 702 detect the movement of joints by monitoring the electrogoniometers 701. The force platforms 703, recessed into the floor of the system, measure the amount of force each foot applies to the ground. The sensors 704, fixed to the shoe soles, measure the distribution of pressure beneath various parts of the foot. An amplifier unit connects the measuring equipment with the processing unit 705.

It is hereby contemplated that the infrared cameras 702, the force platforms 703, and the shoe sensors 704 transmit the detected data to the processing system 705. The processing system 705 reconstructs the gait graphically in 3D visual form and determines the kinematics, joint angle/position changes, joint movement and powers, and extended and undersized bones. The processed data is displayed on the display panel. Using the processed data, a medical practitioner can make a gait characterization, taking into account the patient's posture, balance, stability, symmetry, and foot support pattern.

Once the patient's T-score has been derived and, optionally, gait has been characterized, a patient may require a determination of bone turnover. Determinations of bone turnover rates are performed utilizing conventional serum and/or urine laboratory tests, including fasting calcium/creatinine, hydroxyproline, alkaline phosphatase and/or osteocalcin/bone growth protein. Bone erosion markers, measured in urine, include deoxypyridinoline collagen crosslinks (DPD), N-telopeptides of type 1 bone collagen (NTX), and C-telopeptides of type 1 bone collagen (CrossLaps) and measure breakdown products of bone collagen. Bone formation markers, measured in serum, include osteocalcin and bone specific alkaline phosphatase, which are secreted by osteoblasts (bone forming cells) and indicate the activity of these cells. High levels of bone turnover markers indicate that the patient is a fast bone loser and that the hip fracture risk may be doubled. The lab tests generally utilize standard high pressure liquid chromatography (HPLC) techniques.

Biochemical assessments of bone characteristics can be made by various methods such as enzyme-linked immunosorbent assays (ELISA), radioimmunoassays, immunoradiometric assays, labeled immunoassay technique, capillary electrophoresis technique, western blotting technique, and florescent microscopy technique. Various types of assays such as chemical, enzymatic, immunochemical, and radioimmuno assays may be used on a sample plate to detect the level of markers in the body fluids. For example, chemical assays may detect phosphorous and calcium. Radioimmuno assays can detect radioisotopes such as I¹²⁵, H³, and C¹⁴. Enzymatic assays can detect the action of enzymes such as alkaline phosphatase and pyridoniline. Immunochemical assays may detect biological compounds by monoclonal or polyclonal antibodies or specific receptor proteins. As known by those skilled in the art, several bone specific assays have been developed which enable bone turnover to be evaluated with an immunoassay format.

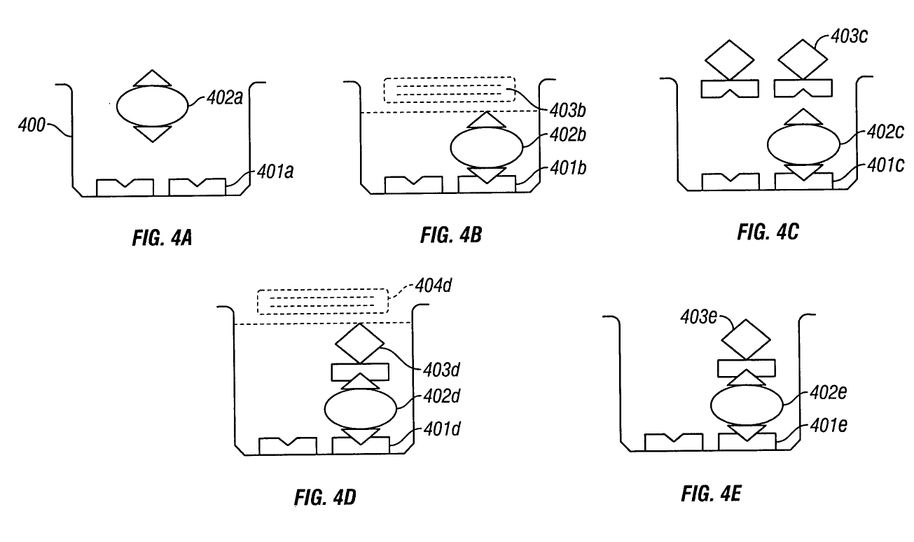

In one embodiment, a labeled immunoassay technique employs a plate containing wells for detecting biochemical markers. Referring to FIG. 4, one method of assaying biomarkers using a plate well 400 is shown. In FIG. 4a antibodies 401a are fixed to the bottom of the well 400. Biomarker samples containing object antigens 402a are introduced to the well. FIG. 4b shows antigen-antibody reaction and each object antigen 402b combines with a solid phase antibody 401b. After antigen-antibody reaction, the liquid layer 403b is removed leaving the combined antigen 401b and antibody 402b. FIG. 4c depicts the effect of introduction of labeled antibodies 403c, such as color reagents, in the well, which combine with object antigens 402c. FIG. 4d depicts antigen-antibody reaction so that the object antigen 402d is sandwiched between the antibodies 401d and 403d. Subsequently, the liquid layer 404d is removed. FIG. 4e shows the well 400 containing labels 403e, which are examined. The number of labels is proportional to the quantity of the object antigens, i.e. biomarkers.

In one embodiment, multiwell plate assays are employed. The plate has antibodies fixed in the wells to capture and detect markers. The antibodies are compatible with the markers to be detected. These antibodies are produced by certain animals in response to an antigen, and are collected, purified, and used as a reagent in immunoassays. The antibodies are pre-applied to the surface of plate wells. Body fluid such as urine or blood is then applied to the surface of the wells. To detect and amplify the initial antigen-antibody reaction in an immunoassay, antibodies must be labeled. Antibodies are labeled using radioisotopes such as I¹²⁵ and H³, fluorescent dyes, such as fluorescein and rhodamine, and enzymes such as horseradish peroxidase (HRP) and alkaline phosphatase (AP). The label on an antibody catalyzes the chemical conversion of a substrate into a product, which can be examined.

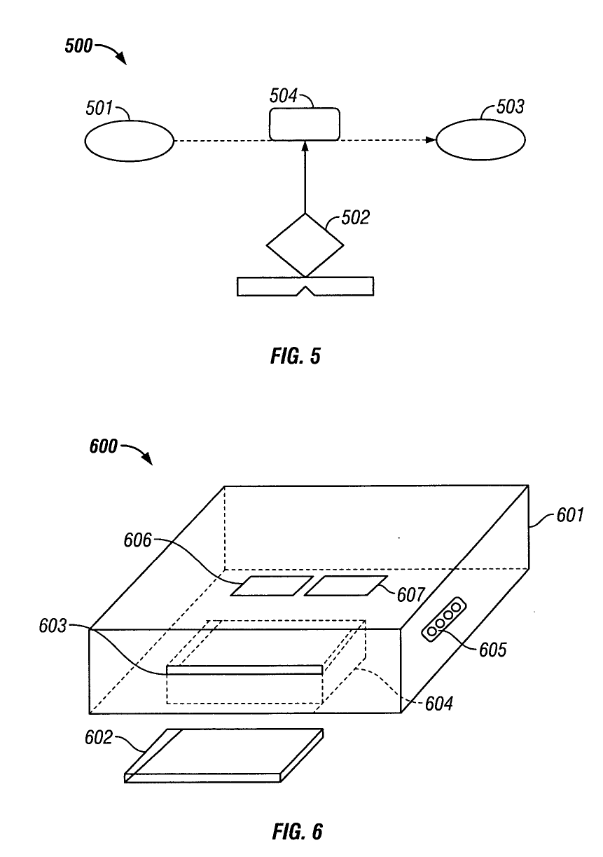

FIG. 5 shows the reaction of a label enzyme with a substrate. The enzyme 501, used as a label, reacts with the antigen-antibody mixture 502 to create the product 503. A photomultiplier tube or a spectrophotometer 504 then detects the florescence or color of the product 503. The extent of color or fluorescent intensity is proportional to the quantity of the biochemical marker.

FIG. 6 shows one embodiment of the bone marker measuring unit. The bone marker measuring unit 600 includes housing 601, sample plate 602, access port 603, plate reader 604, display panel 605, and switches 606 and 607. The access port 603 is designed in such a way so as to receive the sample plate 602 treated with the biochemical marker. The plate reader 604 is built into the housing below the access port 603 and spectrophotometrically measures the optical density or absorbance of the reactions occurring in the plate wells. The plate reader 604 is tuned to a specific wavelength for a particular assay and is used to measure the amount of light absorbed by the reaction of label enzyme with the substrate. The results generated by the plate reader 604 are proportional to the concentration of the absorbing constituent in the solution. The results provided by the plate reader 604 are transmitted to the display panel 605, which displays the bone marker readings. The switch 606 is an ON/OFF switch. The switch 607 is a TEST switch and is used to activate the plate reader to read the sample plate.

In another embodiment of the bone marker measuring unit, a sample of body fluid such as blood or urine is collected in a test tube. The test tube containing the body fluid is placed in an analyzer, which determines the concentration of the bone formation and resorption markers. The concentration of these markers is then compared to the reference values to determine the bone marker levels. This embodiment is particularly useful in determining the bone marker levels on small scale such as laboratories.

Preferably, all of the tests, including the bone scan, gait analysis, and bone marker tests, are performed at the point of care. Specifically, it is preferred that a health care provider can conduct a set of test and provide a patient with a specific set of therapies, recommendations, treatments, or prescriptions prior to the patient leaving the health care provider's premises.

The present invention may optionally use an integrated therapy unit to provide prevention and treatment recommendations based on the diagnosis by the above described bone characteristic measuring, gait analysis, and bone marker measuring units. The treatment recommendations for the prevention and treatment of osteoporosis include life style changes, exercises, calcium and vitamin supplements, and medications. In one embodiment, the integrated therapy unit comprises a receiver, for receiving data outputs from each of the bone characteristic and bone marker measurement units, and the gait analysis technique, a processor for relating the received data outputs to a recommended treatment protocol, set of prescriptions, or other treatments, and a display for displaying such recommendations.

In one embodiment, the treatment recommendations are stored in a data source. The treatment recommendations may be stored in any data structure, including spreadsheet, database or other table formats. In an exemplary use, data is received that indicates the patient's state of bone mass and the gait condition. The processor references a lookup table, in accordance with the data, to determine whether the patient is in a high-risk category. If so, bone marker measurement is then performed to produce marker concentration levels. Based upon the gait characterization, bone density levels, and bone marker levels, or based upon their values relative to a reference level, the processor references the lookup table and retrieves an appropriate protocol particular to the patient's values. Such a protocol is output on the display device as treatment recommendations. These recommendations are then used by practitioners to prescribe treatment regimens and advice patients to comeback for re-examination. One of ordinary skill in the art would appreciate that a plurality of other structural elements would exist in such a processing unit to insure operability, including memory units, data transmission buses, and other data reception, transmission, and processing elements.

One of ordinary skill in the art would also appreciate that data from different examination techniques can be obtained separately and input manually in the integrated therapy unit. Also, the practitioners can analyze the three different types of data manually, corresponding to a patient's values, using the protocols from the lookup tables.

Recommendations can include life style changes such as quitting cigarette smoking and alcohol intake that help in reducing bone loss. Smoking cigarettes can lead to bone weakening. Alcohol consumption is also known to affect bones. Therefore, ceasing alcohol consumption and smoking can help in decreasing bone loss.

Recommendations can also include a proper exercise regimen that helps in building and maintaining normal bone mass and density. Typically, weight bearing and resistance exercises are prescribed. In the weight bearing exercises, bones and muscles work against gravity. Jogging, walking, stair climbing, dancing, racquet sports, and hiking are examples of weight bearing exercises with different degrees of impact. The second type of exercises is resistance exercises that use muscular strength to improve muscle mass and strengthen bone. These activities include weight lifting.

Recommendations can also include a dietary changes that help increase bone mass. A balanced diet rich in calcium and vitamin D helps in preventing bone loss. Depending on the age, an appropriate calcium intake falls between 1000 and 1300 mg a day. Foods such as low-fat milk, cheese, broccoli, orange juice, and cereals are rich in calcium. Calcium supplements in the form of oral pills may also be consumed.

Recommendations can also include increased vitamin intake. Vitamin D plays a major roe in calcium absorption and bone health. It allows calcium to leave the intestine and enter the bloodstream and helps kidneys in resorbing calcium. Vitamin D is manufactured in the skin following direct exposure to sunlight. Usually 10-15 minutes exposure of the body two to three times a week is enough to satisfy the body's vitamin D requirement. The major food sources of vitamin D are vitamin D-fortified dairy products, egg yolks, saltwater fish and liver. Some calcium supplements and most multivitamins also contain vitamin D. Depending on the age, a daily intake of vitamin D between 400 and 800 international units (IU) may be prescribed.

Recommendations can also include the intake of certain medications that positively affect the bone remodeling cycle and are classified as anti-resorptive medications. Anti-resorptive medications slow or stop the bone resorbing portion of the bone-remodeling cycle but do not slow the bone-forming portion of the cycle. As a result, new formation continues at a greater rate than bone resorption, and bone density may increase.

Bisphosphonates such as alendronate and risedronate help in preventing bone loss. Alendronate helps in both the prevention and treatment of osteoporosis by reducing bone loss, increasing bone density and lowering the risk of spine, wrist and hip fractures. A daily dosage of 5 mg for prevention and 10 mg for treatment may be prescribed. Risedronate also helps in the prevention and treatment of osteoporosis by slowing bone loss and reducing the risk of spine and non-spine fractures. A daily dosage of risedronate may be 5 mg per day.

A naturally occurring hormone calcitonin is involved in calcium regulation and bone metabolism in the body. Calcitonin is known for slowing bone loss and increasing spinal bone density while decreasing the rate of bone fractures. Because calcitonin is a protein, it cannot be taken orally because it would be digested before it could work. A daily dosage of 50-100 IU as an injection or 200 IU as nasal spray may be prescribed.

Estrogen replacement therapy (ERT) or hormone replacement therapy (HRT) can also be prescribed for prevention and management of osteoporosis. ERT reduces bone loss, increases bone density, and reduces the risk of hip and spinal fractures. ERT is administered commonly in the form of a pill or skin patch. Raloxifene is another drug that can be administered for the prevention and treatment of osteoporosis.

One of ordinary skill in the art would appreciate that various modifications could be made to the above constructions without departing from the scope of the invention. It is intended that all the matter contained in the above description should be interpreted as illustrative and not in a limiting sense. For example, other configurations of bone densitometers, biochemical analyzers, gait analysis apparatus, or prevention therapies could be used while still staying within the scope and intent of the present invention.

1. A method for treating a patient for a bone related condition comprising the steps of:
measuring a bone characteristic level in a bone of said patient to yield a first score having a value;
conducting a gait analysis on said patient to yield a first gait characterization;
measuring a bone marker concentration in at least one body fluid of said patient to yield a first bone marker level having a value; and
prescribing a therapy based on at least one of said first score, said first gait characterization and said bone marker level value.

2. The method of claim 1 wherein the bone characteristic level is measured using a bone characteristic measuring unit, comprising:
a space for housing a portion of said patient;
a positioning device for holding said portion;
a plurality of ultrasound transducers for transmitting and detecting signals; and
an output for outputting said first score value.

3. The method of claim 2 wherein the bone characteristic is a quantitative ultrasound index.

4. The method of claim 2 wherein the bone characteristic is a stiffness index.

5. The method of claim 1 wherein the bone characteristic level is measured using X-ray absorptiometry.

6. The method of claim 1 wherein the bone characteristic level is measured using quantitative ultrasonometry.

7. The method of claim 1 wherein the bone characteristic level is measured using quantitative computed tomography.

8. The method of claim 1 wherein the bone characteristic is bone mineral density.

9. The method of claim 1 further comprising the step of assessing a plurality of risk factors attributable to the patient.

10. The method of claim 9 wherein said therapy is prescribed based at least in part upon the assessment of said risk factors.

11. The method of claim 1, wherein the first score is a T-score.

12. The method of claim 1 wherein said therapy is prescribed based upon an output of an integrated unit having received the first value, the gait characterization, and the bone marker level value.

13. The method of claim 12, wherein said integrated unit comprises a receiver in data communication with a processing unit and a display unit in data communication with the processing unit.

14. The method of claim 1 further comprising the step of determining a likelihood of said patient injuring one of a plurality of bones of said patient.

15. The method of claim 1 wherein the bone marker level is measured by a bone marker measurement device, wherein said device comprises:
a container containing a body fluid;
a mechanism for holding the said container;
an analyzer for determining a concentration of an absorbing constituent in a solution; and
an output for outputting the first bone marker level value.

16. The method of claim 1 wherein the gait analysis is characterized by a gait analysis procedure conducted on said patient having a balance, wherein said procedure comprises the steps of:
examining the balance of the patient wherein the patient is standing on both feet;
examining the balance of the patient wherein the patient is standing on a first foot; and
examining the balance of the patient wherein the patient is standing on a second foot.

17. The method of claim 1 wherein the gait analysis is characterized by a gait analysis procedure conducted on said patient having a balance, wherein said procedure comprises the steps of:
having the patient stand on a plurality of platforms;
detecting pressure exerted on said plurality of platforms; and
determining a pressure differential on said plurality of platforms.

18. The method of claim 1 wherein said therapy includes at least one of recommending life style changes, recommending weight bearing exercises, and recommending resistance exercises.

19. The method of claim 1 wherein said therapy includes at least one of recommending increasing calcium intake and recommending increasing vitamin D intake.

20. The method of claim 1 wherein said therapy includes recommending at least one of bisphosphonates, calcitonin, estrogen replacement therapy, and raloxifene.

21. The method of claim 1 further comprising the steps of:
within a first pre-defined time period, re-measuring a bone characteristic level in said bone to yield a second score having a value;
within a second pre-defined time period, re-conducting a gait analysis to yield a second gait characterization; and
within a third pre-defined time period, re-measuring a bone marker concentration in the at least one body fluid of said patient to yield a second bone marker level having a value;
comparing the first score to the second score, the first gait characterization to the second gait characterization, and the first bone marker level to the second bone marker level, and;
prescribing a therapy based upon at least one of said comparisons.

22. The method of claim 21 wherein said first, second and third pre-defined time periods are different periods of time.

23. The method of claim 1 wherein a plurality of bone characteristic levels are measured from a plurality of bones of said patient.

24. The method of claim 1 wherein the step of prescribing a therapy is based on said measurement of a bone characteristic level, said gait analysis, and said measurement of a bone mass marker concentration.

25. The method of claim 1 further including the step of designating a future time to repeat said measurement of a bone characteristic level, said gait analysis, and said measurement of a bone mass marker concentration.

26. The method of claim 25 wherein said future time to repeat said measurement of a bone characteristic level is during the twelfth month from the previous measurement.

27. The method of claim 25 wherein the step of designating a future time to repeat said gait analysis includes scheduling a series of eight gait analyses over a period of time.

28. The method of claim 25 wherein said future time to repeat said gait analysis is between one and four months from the previous analysis.

29. The method of claim 25 wherein said future time to repeat said gait analysis is once a week for eight consecutive weeks.

30. The method of claim 25 wherein said future time to repeat said gait analysis is once every two weeks for sixteen consecutive weeks.

31. The method of claim 25 wherein said future time to repeat said bone marker measurement is between two to four months.

32. The method of claim 25 wherein said future time to repeat said bone marker measurement is during the third month from the previous measurement.

33. The method of claim 1 wherein said steps of measuring a bone characteristic level, conducting a gait analysis and measuring a bone marker concentration may be performed in any order.

34. The method of claim 1 wherein said step of conducting a gait analysis is based on the value of said first score.

35. A system for treating a patient for a bone related condition comprising:
a bone characteristic measurement unit having an output for communicating a bone characteristic level value;
a gait analysis unit having an output for communicating a gait characterization; and
a bone marker measurement unit having an output for communicating a bone marker level value.

36. The system of claim 35 wherein said bone characteristic measurement unit comprises a space for housing a portion of said patient, a positioning device connected to said chamber for holding said portion, a plurality of ultrasound transducers for transmitting and detecting signals, and an output for outputting the bone characteristic level value.

37. The system of claim 35 wherein the gait analysis unit comprises at least two pressure sensitive platforms.

38. The system of claim 35 wherein the bone characteristic measurement unit is a X-ray absorptiometry unit.

39. The system of claim 35 wherein the bone characteristic measurement unit is a quantitative ultrasonometry unit.

40. The system of claim 35 wherein the bone characteristic measurement unit is a quantitative computed tomography unit.

41. The system of claim 35 wherein the bone marker measurement unit comprises a container containing a body fluid, an analyzer for determining a concentration of an absorbing constituent in a solution, and an output for outputting the bone marker level value.

42. The system of claim 35 further comprising an integrated unit in data communication with a processing unit for outputting a recommendation, wherein said integrated unit is in data communication with the outputs of said bone characteristic measurement unit, said gait analysis unit, and said bone marker measurement unit, wherein said recommendation is determined by the processing unit based upon the bone characteristic level value, gait characterization, and bone marker level value.

43. A method for treating a patient for a bone related condition comprising the steps of:
instructing a medical practitioner to measure a bone characteristic level in at least one of said plurality of bones of said patient to yield a score having a value;
based upon the value of said score, instructing the medical practitioner to conduct a gait analysis of said patient to yield a gait characterization;
based upon the value of said score and the said gait characterization, instructing the medical practitioner to measure a bone marker concentration in at least one body fluid of said patient to yield a bone marker level having a value;
providing the medical practitioner with a plurality of therapies that can be prescribed; and
instructing the medical practitioner to designate a future time to repeat said measurement of a bone characteristic level, said gait analysis, and said measurement of a bone marker concentration.

44. A method for treating a patient for a bone related condition comprising the steps of:
measuring a bone characteristic level in a bone of said patient to yield a T-score having a value;
if the T-score is abnormal, conducting a gait analysis to yield a gait characterization;
if the gait characterization is abnormal, measuring a bone marker concentration in at least one body fluid of said patient to yield a bone marker level having a value;
prescribing a therapy based on at least one of the said gait characterization, said T-score, and bone marker level; and
designating a future time to repeat said measurement of a bone characteristic level, said gait analysis, and said measurement of a bone marker concentration.

45. The method of claim 44 wherein said future time to repeat said measurement of a bone characteristic level is during the twelfth month from the previous measurement.

46. The method of claim 44 wherein the step of designating a future time to repeat said gait analysis includes scheduling a series of eight gait analyses over a period of time.

47. The method of claim 44 wherein said future time to repeat said bone marker measurement is during the third month from the previous measurement.
